(19) United States
(12) Patent Application Publication
(10) Pub. No.: US 2026/0076817 A1
(43) Pub. Date: Mar. 19, 2026

(54) LOAD-DISTRIBUTION DEVICE FOR ISOLATING ANATOMICAL REGION OF INTEREST

(71) Applicants: Taylor Paziuk, Cleveland, OH (US); John Mangan, Drexel Hill, PA (US); Andrew J. Miller, Haddonfield, NJ (US); Justin Stull, Ho-Ho-Kus, NJ (US)

(72) Inventors: Taylor Paziuk, Cleveland, OH (US); John Mangan, Drexel Hill, PA (US); Andrew J. Miller, Haddonfield, NJ (US); Justin Stull, Ho-Ho-Kus, NJ (US)

(21) Appl. No.: 18/886,029

(22) Filed: Sep. 16, 2024

(51) Int. Cl. A61F 5/30

(52) U.S. Cl. CPC A61F 5/30

(57) ABSTRACT

A load distribution device is configured to be placed against a region of interest of a patent that is susceptible to damage when placed against a support surface for a prolonged period of time. The load distribution device is placed against the patient such that the region of interest is aligned with an aperture of the load distribution device. The load distribution device can surround the region of interest. Thus, anatomical loading that might otherwise act against the region of interest instead is redistributed to a region of the patent that is adjacent the region of interest, and aligned with the body of the load distribution device.

### BACKGROUND

Surgical procedures typically involve the involve creating an incision in an epidermis of the patient, creating a conduit to a target location, performing at least one surgical operation at the surgical location, removing the surgical implements, and closing the surgical incision. After completion of the surgery, the patient typically lays in a supine position against a support surface, which can be defined by a bed or cot or the like, thereby causing the closed incision to bear against the support surface under an anatomical load that is produced under the weight of the patient. Conventional devices exist that redirect loads from the support surface away from the surgical wound. For example, U.S. Pat. No. 10,548,790 describes a post-surgical support member that is applied to the patient and is configured to offload forces from a support surface to tissue that has been subjected to surgical wounds.

While this device is suitable for its intended purpose, other challenges exist. For instance, positioning sedated, unconscious, and/or physically unaware patients against a support surface often places vulnerable tissue and boney prominences at risk owing to a lack of cognizant sensory feedback from the patients themselves during the positioning and/or application process. Accordingly, the pressure applied to these vulnerable areas of the patient by the support surface over long durations can result in significant morbidity to these areas of the patient. Because the patient is unable to sense discomfort and reposition themselves, the patient is unable to prevent tissue morbidity of pressure induced injury. Similarly, because the patent is unable to provide feedback of discomfort, the caregiver lacks the information necessary to take remedial action.

Conventional approaches aimed at addressing this issue fall short in several aspects. For instance, they are typically limited in their degrees of freedom by the positioning apparatus rather than the patients themselves. Conventional approaches that are designed to address this issue from a patient specific standpoint typically only function to uniformly distribute pressure over a vulnerable site of interest and decrease the shear stress on the overlying skin rather than decreasing the pressure over the vulnerable tissue.

What is desired is a device that is configured to proactively decrease pressure from a support surface to vulnerable patent sites of interest to prevent the formation of pressure wounds.

### SUMMARY

The following Summary is provided to introduce a selection of concepts in a simplified form that are further described below in the Detailed Description. This Summary is not intended to identify key features or essential features of the invention, nor is it intended to be used to limit the scope of the invention. Reference is made to the claims for that purpose.

In one example, a method is provided for isolating anatomical forces of an identified vulnerable region of interest of a patient from a support surface. The method can include the step of placing a load distribution device between the patient and the support surface such that an entirety of the region of interest is aligned with an aperture that extends at least into a body of the load distribution device. The placing step can include aligning the body with an adjacent support location that is adjacent the region of interest. The method can further include the step of iterating the patient to a position against the support surface such that the load distribution device isolates anatomical forces of the patient between the region of interest and the support surface.

### BRIEF DESCRIPTION OF THE SEVERAL VIEWS OF THE DRAWINGS

The foregoing summary, as well as the following detailed description, is better understood when read in conjunction with the appended drawings. There is shown in the drawings example embodiments, in which like reference numerals correspond to like reference numerals throughout. The present invention is not intended to be limited to the specific embodiments and methods disclosed, and reference is made to the claims for that purpose.

FIG. 1A is a schematic illustration of a patient in a supine position supported by a support surface, shown with load distribution devices applied to regions of interest of the patient;

FIG. 1B is a schematic illustration of a patient in a lateral position supported by a support surface, shown with load distribution devices applied to regions of interest of the patient;

FIG. 1C is a schematic illustration of a patient in a prone position supported by a support surface, shown with load distribution devices applied to regions of interest of the patient;

FIG. 1D is a schematic illustration of a patient in a lateral position supported by a support surface, shown with load distribution devices applied to regions of interest of the patient;

FIG. 1E is a schematic illustration of a patient in a lateral position supported by a support surface, shown with load distribution devices applied to regions of interest of the patient;

FIG. 2 is an elevation view of a posterior aspect of the patient illustrated in FIG. 1, showing a plurality of load distribution devices applied to various regions of interest of the patient;

FIG. 3A is a perspective view of a load distribution device applied to and conforming to a region of interest;

FIG. 3B is a sectional elevation view of the region of interest of the patent illustrated in FIG. 3A, showing the load distribution device disposed on a support surface under anatomical loading;

FIG. 4A is a perspective view of the load distribution device illustrated in FIGS. 3A and 3B, but shown in an initial configuration;

FIG. 4B is a side elevation view of the load distribution device illustrated in FIG. 4A;

FIG. 4C is a top plan view of the load distribution device illustrated in FIG. 4A;

FIG. 4D is a top plan view of the load distribution device illustrated in FIG. 4C, but shown including perforated regions in another example;

FIG. 5A is a sectional side elevation view of the load distribution device in one example, showing one of a plurality of separation zones configured as an opening;

FIG. 5B is a sectional side elevation view of the load distribution device similar to FIG. 5A, but showing the separation zones configured as perforated regions;

FIG. 5C is a sectional side elevation view of the load distribution device of FIG. 5B, but showing but showing load distribution device in a conformed configuration;

FIG. 6A is a top plan view of the load distribution device constructed in accordance with another example;

FIG. 6B is a top plan view of the load distribution device of FIG. 6A constructed in accordance with yet another example;

FIG. 7A is an elevation view of a posterior aspect of the patient showing a wound; and

FIG. 7B is an elevation view of the posterior aspect of the patient of FIG. 7A, showing a load distribution device applied to a region of the patent that defines the wound.

### DETAILED DESCRIPTION

Disclosed is a load distribution device that is configured to be directly applied to the patient adjacent a vulnerable anatomical region of interest that is expected to be subjected to prolonged pressure, for instance from a support surface. The load distribution device redirects and diffuses loads from the support surface that would otherwise have acted on the region of interest to locations adjacent the region of interest. Because the device is applied directly to the patient, it is not limited by a predetermined positioning bed or other apparatus. The device decreases the pressure to vulnerable tissue at the region of interest, thereby reducing or minimizing the pressure over a vulnerable area and distributing it more uniformly to the surrounding tissue that can accommodate these stressors. Accordingly, tissue morbidity due to prolonged applied pressure at regions of interest can be reduced or eliminated.

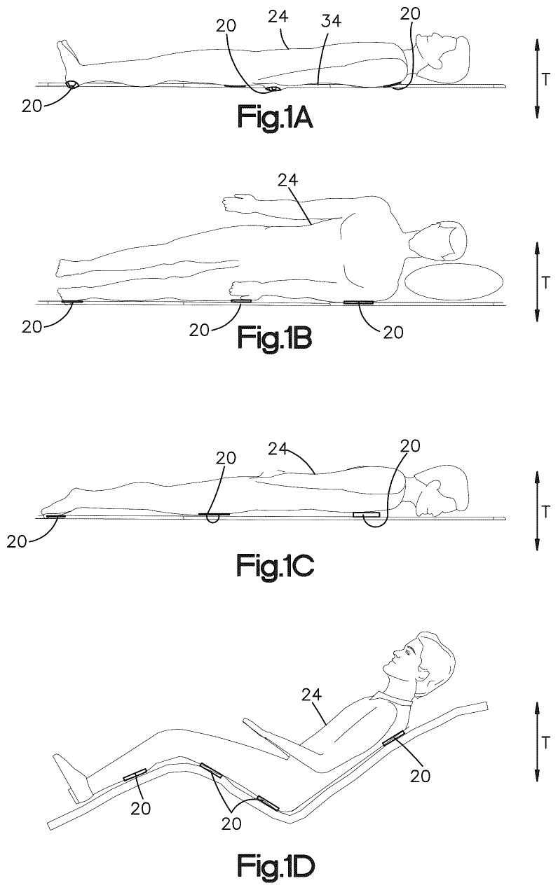

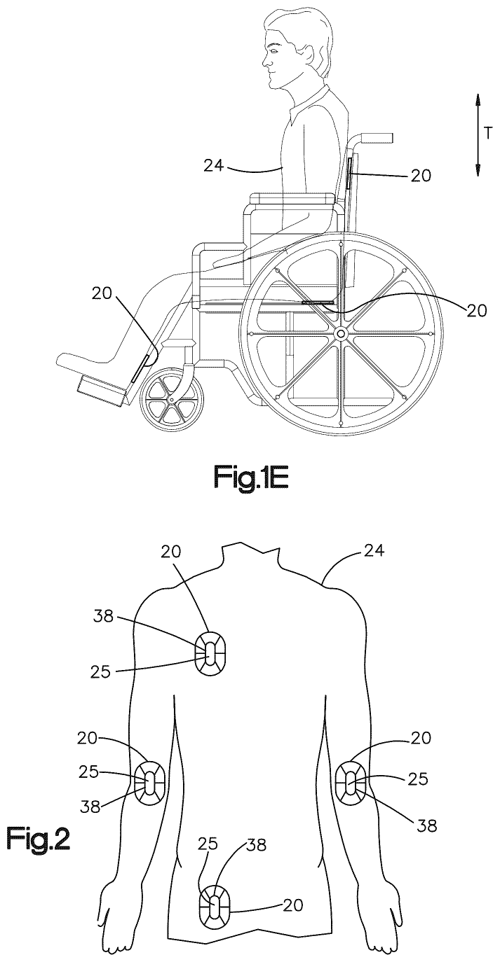

Referring to FIGS. 1A-3, a load distribution device 20 is configured to support an anatomical region of interest 25 of a patient 24 that is supported by a support surface 34. For instance, the patient can undergo a surgical procedure. Alternatively, the patient can be on bedrest. Alternatively, the patient can be wheelchair bound. Thus, the support surface 34 can be defined by a bed, a gurney, a couch, a chair, or any suitable structure as desired. The present inventors recognize that when a patient is supported on the support surface 34 in a supine position (FIG. 1A), in a lateral position (FIG. 1B), seated in a conventional chair (FIG. 1C), reclined (FIG. 1D), in a wheelchair (FIG. 1E), or otherwise generally stationary on a support surface for a long period of time, the patient can define one or more lead-bearing regions of interest 25 of his or her body that bears against the support surface that are susceptible to pressure wounds. In particular the weight of the patient at the regions of interest can cause the support surface to exert an anatomical force to the regions of interest 25. Further, the patient's muscles can exert a bias to the region of interest against the support surface 34. The locations of the regions of interest 25 can differ depending, for instance, on the position of the patient 24 and the nature of the support surface 34. When the patient is under sedation, unconscious, physically unaware, or otherwise has diminished senses and/or diminished ability to communicate with a caregiver, the caregiver will be unaware that the patient should be repositioned at various time intervals to prevent or limit morbidity to the patient's tissue at the regions of interest 25. As shown at FIGS. 1A-2, the caregiver can apply one or more load distribution devices 20 to a corresponding one or more regions of interest 25 to prevent pressure wounds, which can include tissue and/or nerve damage.

By way of example and not limitation, the anatomical regions of interest 25 can be defined by one or more anatomical non-flat surfaces or prominences, such as one or more bony prominences and/or one or more epicondyles that are weight bearing against the support surface 34. Even though the nominal weight of the region of interest 25 may not be high, the pressure per square inch (PSI) can be dangerous due to the prominence of the region of interest 25. Left unaddressed, the PSI acting on the region of interest 25 for a prolonged period of time can create sores, nerve damage, and other potential tissue damage.

The boney prominences can be defined by either or both of the anterior superior iliac spine (ASIS) and the iliac crest during prone or lateral positioning against the support surface 34. Alternatively or additionally, the boney prominence can be defined by any one or more up to all of the posterior superior iliac spine (PSIS), sacrum, and iliac crest during supine or lateral positioning against the support surface 34. Alternatively or additionally, the boney prominence can be defined by either or both of the manubrium and the sternum during prone positioning against the support surface 34. Alternatively or additionally, the bony prominence can be defined by the medial epicondyle of the elbow and associated traversing ulnar nerve during prone, supine, or lateral positioning against the support surface 34. Alternatively or additionally, the bony prominence can be defined by the knee during prone positioning against the support surface 34. Alternatively or additionally, the bony prominence can be defined by the fibular head and traversing peroneal nerve during lateral positioning against the support surface 34. Alternatively or additionally, the bony prominence can be defined by the medial malleolus of the ankle and associated traversing tibial nerve during prone, supine, or lateral positioning against the support surface 34. Alternatively or additionally, the bony prominence can be defined by the lateral malleolus of the ankle and associated traversing superficial peroneal nerve during prone, supine, or lateral positioning against the support surface 34. Alternatively or additionally, the bony prominence can be defined by the calcaneus during prone, supine, or lateral positioning against the support surface 34.

As described above, the regions of interest 25 can be defined by one or more epicondyles. For instance, the epicondyle can be defined by the lateral epicondyle of the elbow during prone, supine, or lateral positioning against the support surface 34.

In still other examples, the regions of interest 25 can be defined during and throughout splint application and wearing. For instance, the regions of interest 25 can be defined by a boney prominence of the lateral malleolus of ankle and associated traversing superficial peroneal nerve during and throughout splint application and wearing. Alternatively or additionally, the bony prominence can be defined by the calcaneus during and throughout splint application and wearing. Alternatively or additionally, the regions of interest 25 can be defined by a medial epicondyle of the elbow and associated traversing ulnar nerve during and throughout splint application and wearing. Alternatively or additionally, the regions of interest 25 can be defined by the lateral epicondyle of elbow during and throughout splint application and wearing. Alternatively or additionally, the bony prominence can be defined by the fibular head and traversing peroneal nerve during and throughout splint application and wearing. Alternatively or additionally, the bony prominence can be defined by the medial malleolus of ankle and associated traversing tibial nerve during and throughout splint application and wearing.

As will be appreciated from the description below, each of the load distribution devices can be applied to the patient adjacent a respective one of the regions of interest so as to distribute the anatomical loads from the support surface away from the regions of interest. In particular, the load distribution device 20 can have a thickness that is suitable to maintain the region of interest 25 at a location spaced above the support surface 34 along a transverse direction T. As a result, the load distribution device 20 is configured to isolate the anatomical force of the patient 24 between the region of interest 25 and the support surface 34. The force can be defined by either or both of the weight of the patient 24 at the region of interest 25 against the support surface 34 along with any muscular-induced bias of the region of interest 25 toward the support surface 34. Thus, the anatomical load does not travel from the support surface 34 to the region of interest along the transverse direction T.

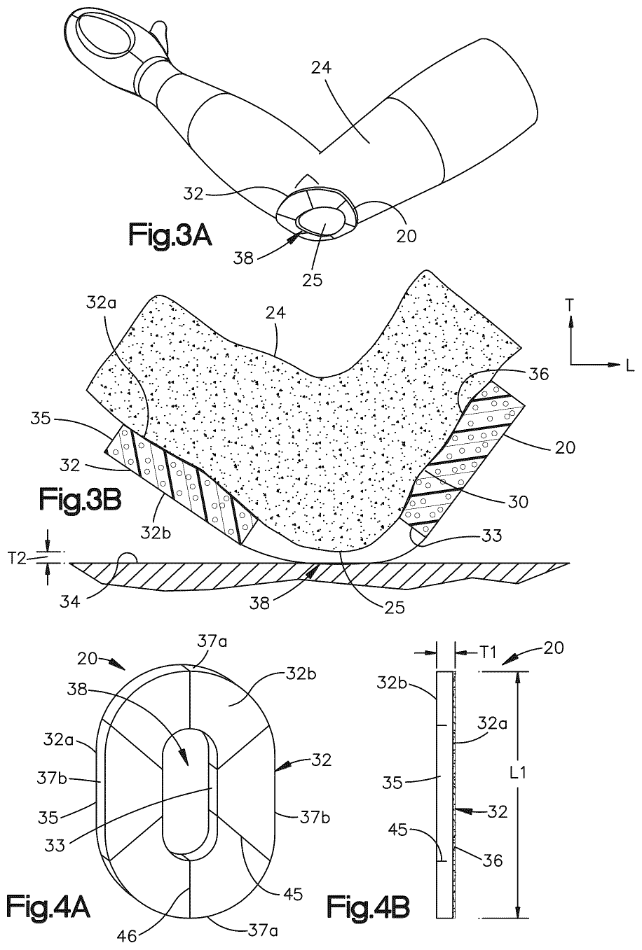

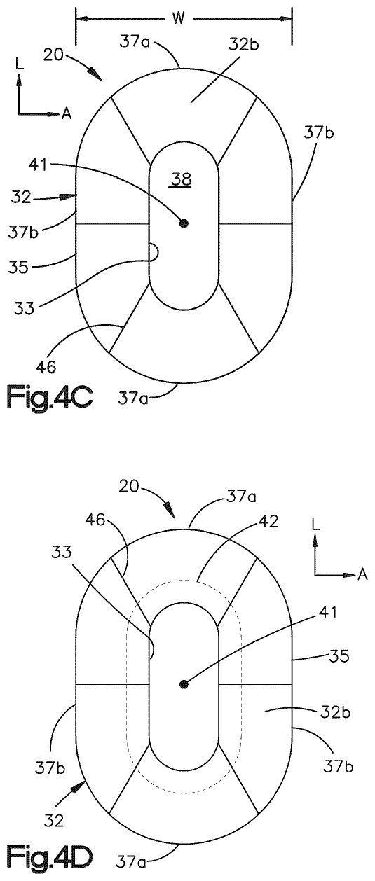

Referring now to FIGS. 3A-4C, the load distribution device 20 includes a body 32 that defines a first or inner surface 32a and a second or outer surface 32b opposite the first surface 32a along the transverse direction T. The first surface 32a defines a patient-facing surface that is configured to face or otherwise be placed against the epidermis 22 or outer anatomical layer of the patient 24 at an adjacent support location 30 that is adjacent the region of interest 25. For instance, the adjacent support region 30 can surround the region of interest 25. In particular, the adjacent support region 30 can be defined by the epidermis 22 of the patient 24 that surrounds the region of interest 25. The body 32 can define an exterior surface 35 that defines an outermost perimeter of the load distribution device that extends from the first surface 32a to the second surface 32b. In one example, the body 32 of the load distribution device 20 can have a hardness less than the hardness of the support surface 34. For instance, the body 32 of the load distribution device 20 can be compressible so as to provide comfort to the patient 24 as the body 32 is compressed along the transverse direction T by the anatomical loads. The body 32 can be compressible along the transverse direction T from a first or relaxed configuration to a second or compressed configuration. The force of the patient at the anatomical region of interest 25 and the adjacent support location 30 can cause the body 32 to compress from the first configuration to the second configuration. Thus, the first and second surfaces 32a and 32b are spaced from each other along the transverse direction to define a first thickness T1 when the load distribution device 20 is in the relaxed configuration, and the first and second surfaces 32a and 32b are spaced from each other along the transverse direction to define a second distance T2 that is less than the first thickness T1 when the load distribution device 20 is in the compressed configuration.

The first thickness T1 and the second thickness T2 can be as desired. By way of example, the relaxed first thickness T1 of the load distribution device 20 can be between approximately 0.25 inch and approximately 6 inches, such as between approximately 0.5 inch and 2 inches, such as between approximately 0.5 inch and approximately 1 inch. In one example, the first thickness T1 can be approximately 0.75 inch. It should be appreciated that the term “approximately,” “about,” and “substantially” and derivatives thereof regarding a parameter includes the stated value of that parameter including plus or minus 10%, including 9%, including 8%, including 7%, including 6%, including 5%, including 4%, including 3%, including 2%, including 1% of the stated parameter. It is recognized that larger regions of interest 25 can benefit from an increased first thickness T1.

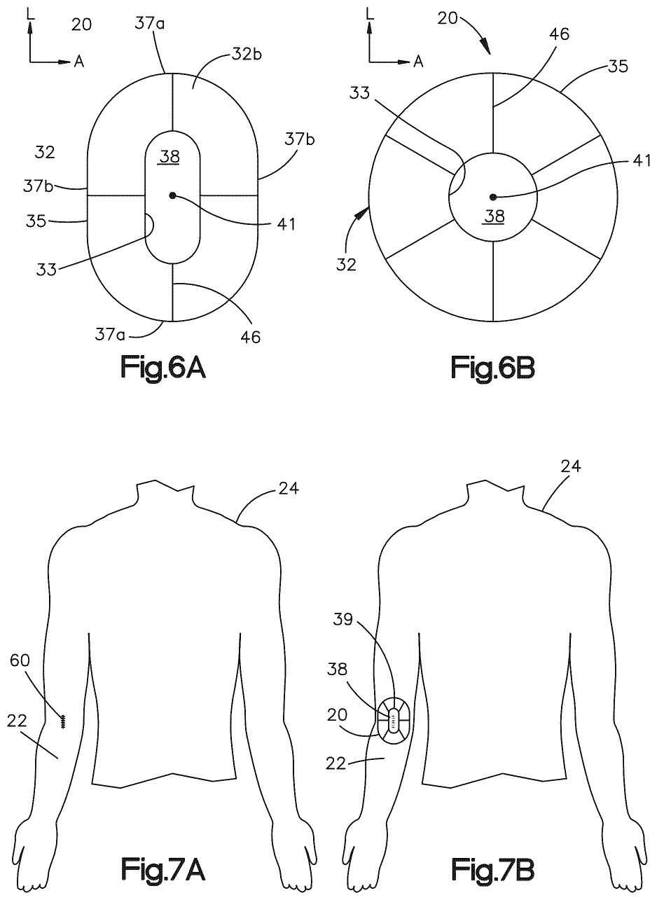

The body 32 of the load distribution device can define an aperture 38 that extends along the transverse direction T at least into the first surface 32a. Thus, the body 32 can define an annulus having the inner aperture 38. In some examples, the inner aperture 38 can be a central aperture of the body 32. A geometric center 41 of the body 32 can also define a geometric center of the inner aperture 38. The body 32 can define an interior surface 33 that defines the perimeter of the aperture 38 with respect to a plane that is defined by the longitudinal direction L and the lateral direction A when the body 32 is in a relaxed state. The interior surface 33 can be opposite the exterior surface 35. Thus, the body 32 can extend from the interior surface 33 to the outer perimeter 35. In one example, the body 32 can extend radially outward from the interior surface 33 to the outer perimeter 35. In this regard, the interior surface 33 and the exterior surface 35 can each define a racetrack shape as illustrated in FIG. 4C. Thus, the body 32 can define curved regions 37a at respective ends that are opposite each other along a longitudinal direction L that is perpendicular to the transverse direction T, and straight sides 37b that are opposite each other along a lateral direction A that is perpendicular to each of the longitudinal direction L and the transverse direction T, and extend to the respective curved regions 37a. In other examples, the interior surface the interior surface 33 and the exterior surface 35 can each define respective circles (see FIGS. 6A-6B). Thus, it should be appreciated that a respective portion or entirety of either or both of the interior surface 33 and the exterior surface 35 can be curved in a plane that is oriented perpendicular to the transverse direction T. Alternatively, the body 32 can define any suitable shape as desired, including oval, square, rectangular, triangular or any suitable alternative regular or irregular geometry. Further, the interior surface 33 and the exterior surface 35 can be parallel to each other. Alternatively, the interior surface 33 and the exterior surface 33 can be non-parallel to each other.

The aperture 38 can extend from the first surface 32a along a direction toward the second surface 32b. In one example, the aperture 38 can extend entirely through the body 32 from the first surface 32a to the second surface 32b. When the load distribution device 20 supports the patient at a location adjacent the region of interest 25, also referred to as an adjacent support location 30 of the patient 24, at least a portion of the aperture 38 can be aligned with the region of interest 25 along the transverse direction T. For instance, an entirety of the region of interest 25 can be aligned with the aperture 38. Because the body 32 is disposed between the adjacent support location 30 and the support surface 34, and the region of interest is aligned with the support surface 34 and the aperture 38 along the transverse direction T, the body 32 can maintain the region of interest at a location spaced from the support surface 34 along the transverse direction. Thus, the load distribution device 20 can isolate and redistribute the anatomical loads produced by the force of the patient 24 against the support surface 34 at the region of interest 25.

The first thickness T1 can be selected such that the region of interest 25 remains spaced from the support surface 34, or significantly de-loaded from the support surface 34, when the load distribution device 20 is compressed to the second thickness T2. In other words, the first thickness T1 can be selected such that the region of interest 25 projects into the aperture 38 toward the second surface 32 and terminates without crossing the second surface 32b (see FIG. 3B). Thus, the region of interest 25 is devoid of direct pressure from the support surface 34 because the body 32 can encircle the region of interest 25 and deliver the anatomical forces to the adjacent support location 30.

In another example, the aperture 38 can extend from the first surface 32a toward the second surface 32b along the transverse direction T, but terminates at a base that is disposed between the first surface 32a and the second surface 32b with respect to the transverse direction T. The base can be spaced from the first surface 32a a sufficient distance along the transverse direction T such that the region of interest 25 is spaced above the base when the load distribution device 20 supports the patient 24 at the adjacent support location 30. Thus, the load distribution device 20 can isolate the entirety of the anatomical load produced from force of the patient 24.

Alternatively, the base can be spaced from the region of interest 25 before the force of the patient 24 bears against the load distribution device 20, but can contact the region of interest 25 when the force of the region of interest 25 that is supported by the load distribution device 20 cause the load distribution device to compress. In this example, the load distribution device 20 isolates a portion of the anatomical loads produced by the force of the patient at the region of interest 25. It can thus be said that the load distribution device 20 can isolate at least a portion of the force of the anatomical loads produced from the force of the patient 24 of the region of interest 25 and the adjacent support location 30 against the support surface 34 when the load distribution device 20 is disposed between the adjacent support location 30 and the support surface 34 at the adjacent support location 30.

The first surface 32a can be substantially planar along a respective plane defined by the longitudinal direction L and the lateral direction A when the load distribution device 20 is in the first configuration. Similarly, the second surface 32b can be substantially planar along a respective plane defined by the longitudinal direction L and the lateral direction A when the load distribution device 20 is in the first configuration. Thus, the first and second surfaces 32a and 32b can be oriented substantially parallel to each other when the load distribution device 20 is in the first configuration. It should be appreciated, of course, that the first and second surfaces can have any orientation as desired such that the load distribution device 20 is configured to isolate the anatomical loads between the anatomical region of interest 25 and the support surface 34.

The first surface 32a is configured to face the epidermis 22 of the patient 24 during operation. In particular, the first surface 32a is configured to face the adjacent location that is disposed outward from the region of interest 25 along the epidermis 22. At least a portion up to an entirety of the adjacent support location 30 can be defined by the epidermis 22. Thus, the first surface 32a is configured to provide physical support to the adjacent support location 30 when the body 32 is disposed between the support surface 34 and the epidermis 22 of the patient 24. In one embodiment, the first surface 32a can be configured to adhesively attach to the adjacent support location 30. It may be preferable in certain circumstances that the first surface 32a attaches to the epidermis 22 at the adjacent support location 30 so as to facilitate easy removal of the load distribution device 20 without affecting the integrity of the site of interest 25.

The second surface 32b is configured to face the support surface 34. Thus, it should be appreciated that the first and second surfaces 32a and 32b are configured to be disposed between the epidermis 22 and the support surface 34. It is recognized that an intermediate structure can be disposed between the second surface 32b and the support surface 34. In one example, the second surface 32b is configured to abut the support surface 34. Because the load distribution device 20 spaces the region of interest 25 from the support surface 34, the load distribution device 20 can be referred to as a spacer. Similarly, the body 32 of the load distribution device 20 can be referred to as a spacer body. As will now be described, when the load distribution device 20 is disposed between the support surface 34 and the patient 24, the load distribution device 20 receives anatomical loads produced from the force of the patient 24 against the support surface 34, thereby isolating the anatomical loads from the region of interest 25.

In one example, the body 32 can be configured to at least substantially surround or entirely surround the region of interest 25. The load distribution device 20 is thus configured to be disposed between the adjacent support location 30 of the epidermis 22 and the support surface 34 when the patient 24 bears against the support surface 34 for a prolonged period of time that could otherwise be sufficient to cause morbidity at the region of interest 25. Thus, a load produced by the force of the patient 24 that would otherwise bear directly on the region of interest 25 instead bears against the load distribution device 20 at the adjacent support location 30.

Further, the body 32 of the load distribution device 20 can be porous with respect to airflow through the body along a direction that is perpendicular to the transverse direction T, such that the load distribution device 20 can allow ambient air to access the region of interest 25. In one example, the body 32 of the load distribution device 20 can be made from a hypoallergenic or non-allergenic material. In one example, the body 32 can be made from a foam material. In a further example, the body 32 of the load distribution device 20 can be a memory foam. For instance, the body 32 can be a polyurethane-based memory foam. As one example, the polyurethane-based memory foam can be a Capu-Cell® Polyurethane Foam, commercially available from TMP Technologies, Inc., having a place of business at 1200 Northland Avenue, Buffalo, NY 14215. Alternatively, the body 32 can be a silicone-based memory foam or any suitable alternative material.

In another example, the body 32 can be a viscoelastic polyurethane, or any suitable alternatively constructed memory foam. The body 32 of the load distribution device 20 can have any suitable porosity as desired. The porosity can be defined as a density that is measured in terms of pounds per cubic foot. It should be appreciated that the porosity of the body 32 can allow for breathability at the surgical site 27 without compromising the durability of the body 32 when the body 32 is compressed by the anatomical loads. In one example, the body 32 can have a density in a range that has a lower end and an upper end. The lower end of the range can be approximately 0.5 pounds per cubic foot, or alternatively approximately 2 pounds per cubic foot, and the upper end of the range can be approximately 10 pounds per cubic foot, or alternatively approximately 8 pounds per cubic foot. For instance, the range can include a lower range that is between and includes approximately 1.5 pounds per cubic foot and approximately 3 pounds per cubic foot. The range can include a middle range that is between and includes approximately 3 pounds per cubic foot and approximately 6 pounds per cubic foot. The range can further include an upper range that is between and includes approximately 6 pounds per cubic foot and approximately 10 pounds per cubic foot. It should be appreciated that the density is provided by way of example, only, and that the body 32 can have any suitable density as desired without departing from the present disclosure.

Referring now to FIGS. 3A-5C, the first surface 32a can be configured to abut the adjacent support location 30. Thus, the body 32, and in particular the first surface 32a, can mold or conform to the epidermis 22 of the patient 24 at the adjacent support location 30, such that the aperture 38 is aligned with the region of interest 25. In particular, the load distribution member 20 is movable, such as flexible, from a first or initial configuration (FIG. 4A) to a second or deformed configuration (FIG. 3B). In the first or initial configuration, the first and second surfaces 32a and 32b can be substantially (e.g., within tolerance) flat, and oriented substantially (e.g., within tolerance) along respective planes that are perpendicular to the transverse direction T. In the second or deformed configuration, the first surface 32a can be concave in orthogonal planes, and the second surface 32b can be convex in orthogonal planes. Otherwise stated, the load distribution device 20 can increase its curvature as it iterates from the initial configuration toward and to the deformed configuration. When the body 32 is applied to the patient 24 and deformed so as to conform to the patient in the manner described herein, the load distribution device can be said to be in a conformed configuration.

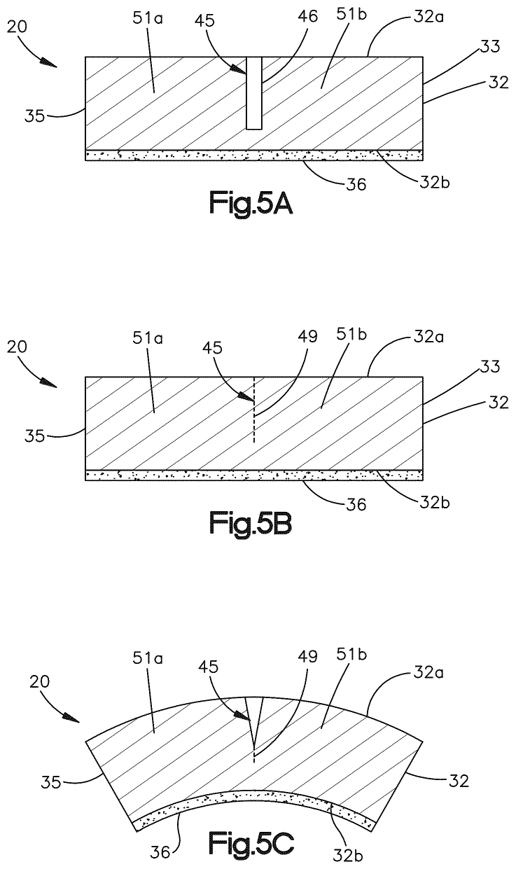

Referring now to FIGS. 5A-5C, in one example the load distribution device 20 can include a plurality of separation zones 45 that are configured to reduce or eliminate buckling of the body 32 as it transitions from the initial configuration to the deformed configuration. The separation zones 45 can assist in conforming the body 32 to the underlying epidermis 22 or outer anatomical layer of the patient 24, particularly when the body flexes along multiple different directions to achieve conformance with the underlying epidermis 22 or outer anatomical layer of the patient 24. Each separation zone 45 defines a respective pair of first and second separation portions 51a and 51b of the body 32 that are separated from each other by the separation zone 45. Further, the first and second separation portions can further separate (i.e., move away) from each other as the load distribution device 20 moves from the initial configuration to the conformed configuration. That is, at least a portion of the separation zone 45 can widen, which causes the first and separation portions 51a and 51b to move away from each other. Each separation zone 45 can be elongate along a respective separation zone axis between the outer surface 35 and the inner surface 33. For instance, each separation zone 45 can extend along a respective separation zone axis from the outer surface 35 to the inner surface 33. Each separation zone axis can be straight and linear in some examples. In other examples, they can be curved or bent. Each separation zone axis can intersect a centerline that is oriented along the transverse direction and extends through the geometric center 41 of the body 32. It should be appreciated, of course, that the separation zones 45 can be oriented in any suitable alternative direction as desired. In one example, the separation zones 45 can be equidistantly spaced from each other about the geometric center 41, or can be variably spaced from each other as desired.

In some configurations, opposed pairs of separation zones 45 can be defined that are aligned with each other and opposite each other with respect to the aperture 38. Aligned opposed pairs of separation zones 45 can be aligned with each other along a common plane that includes the transverse direction T. Thus, the separation zone axes of respective opposite pairs of separation zones 45 can be coincident with each other so as to define a common axis. For instance, when the load distribution device 20 includes an even number of equidistantly spaced separation zones 45, the separation zones 45 can be arranged in a number of opposite pairs of separation zones 45 that is equal to one-half the even number of separation zones 45.

The load distribution device 20 can include any number of separation zones 45 as desired, such as equal to or greater than two separation zones 45, such as equal to or greater than three separation zones 45, such as equal to or greater than four separation zones 45, such as equal to or greater than five separation zones 45, such as equal to or greater than six separation zones 45, equal to or greater than seven separation zones 45, such as equal to or greater than eight separation zones 45, such as equal to or greater than nine separation zones 45, such as equal to or greater than ten separation zones 45.

In one example, the load distribution device 20 can include at least two pairs of aligned separation zones 45 (see FIG. 6A). Thus, the load distribution device 20 can separate along at least two separation zone axes, such as at least two common separation zone axes that are common to the at least two respective pairs of separation zones 45. The separation zone axis can be angularly offset from each other by an amount in a range from about 5 degrees to about 90 degrees, such as from about 45 degrees to about 90 degrees, such as from about 60 degrees to about 90 degrees. In one example, the separation zone axes can be angularly offset from each other by about 90 degrees when the load distribution device 20 includes two pairs of aligned separation zones 45 as shown at FIG. 6A. In other examples, the separation zone axes can be angularly offset from each other by approximately 60 degrees, for instance when the load distribution device includes three pairs of aligned separation zones 45 as shown in FIGS. 4A-4C. In still other examples, the load distribution device 20 can include an odd number of separation zones 45 that can be equidistantly or variably spaced from each other about the body. For instance, the load distribution device 20 can include three separation zones that are equidistantly spaced form each other, and thus angularly offset from each other by approximately 120 degrees. In other examples, the load distribution device 20 can include five separation zones that are equidistantly spaced form each other, and thus angularly offset from each other by approximately 72 degrees.

Referring now to FIG. 5A, in one example the separation zones 45 can be configured as a respective opening 46 such as a plurality of openings that extend into the second surface 32b along the transverse direction T toward the first surface 32a without extending through the first surface 32a. Thus, the openings 46 can terminate at an internal base 47 of the body 32. The openings 46 can extend to any depth along the transverse direction T as desired. In one example, the depth is greater than half the first thickness T1 (see FIG. 4B) from the first surface 32a to the second surface 32b along the transverse direction T. For instance, the depth can be greater than 75% of the first thickness T1. In other examples, depth can be in a range from 5% to 50% of the first thickness T1, such as from 25% to 50% of the first thickness T1.

During use, each opening 46 can widen at as the body 32 flexes about the bases 47 when the first surface 32a conforms to a convex epidermal surface, which can surround the region of interest 25, for instance when the region of interest 25 is defined by a bony prominence. The openings 46 can widen at a greater rate at the second surface 32b compared to at a location adjacent the base 47. The bases 47 can define living hinges of the body 32 as the openings 46 to widen when the load distribution device 20 moves or flexes from the initial configuration to the deformed configuration, thereby allowing the load distribution device 20 to contour to the epidermis 22 at the adjacent support region 30 (see FIG. 2). The widening of the openings 46 allow the respective first and second separation portions 51a and 51b to move away from each other. The openings 46 can each extend from the outer surface 35 of the body 32 to the inner surface 33 of the body 32, and thus can be open to the ambient environment at its outer end, and open to the aperture 38 at its inner end. The separation zone axes of the openings 46 can be oriented along the lateral direction A, the longitudinal direction L, or any suitable direction that is substantially perpendicular to the transverse direction T.

The openings 46 can be pre-fabricated prior to use in one example. For instance, the openings 46 can be defined by cut lines that are produced by laser cutting respective slits into the second surface 32b toward the first surface 32a, and from the outer surface 35 to the inner surface 33. The central aperture 38 can also be laser cut from stock foam. The openings 46 can be elongate along respective axes from the outer surface 35 to the inner surface 33. The axes can be oriented along any suitable directions as desired so long as the openings 46 assist in contouring the load distribution member 20 to conform to the epidermis 22 at the adjacent support locations 30. In one example, the axes can intersect each other at the geometric center 41 of the body 32 as described above. The geometric center 14 of the body 41 can be the geometric center of the outer surface 35, the inner surface 33, or both, in a plane that is oriented perpendicular to the transverse direction T. The load distribution device 20 can have any suitable number of openings 46 as desired, such as three, four, five, six, seven, eight, nine, ten, or greater than ten, such that the openings 46 maintain the structural rigidity of the body 32 while providing for the desired ability for the first surface 32a to conform to the epidermis 22 of the patient 24 at the adjacent support location 30.

Referring now to FIGS. 5B-5C, in another example the separation zones 45 can be defined by perforations that define respective perforated regions 39 of the body 32. The body 32 can be perforated along the respective separation zone axes defined by the perforated regions 49. The perforated regions 49 can extend into the second surface 32b toward the first surface 32a along the transverse direction T without extending through the first surface 32a. Thus, the perforated regions 49 can terminate at a location between the first surface 32a and the second surface 32b. The perforated regions 49 can extend to any depth along the transverse direction T as desired. In one example, the depth is greater than half the first thickness T1 (see FIG. 4B) from the first surface 32a to the second surface 32b along the transverse direction T. For instance, the depth can be greater than 75% of the first thickness T1. In other examples, depth can be in a range from 5% to 50% of the first thickness T1, such as from 25% to 50% of the first thickness T1.

During use, the perforated regions 49 are configured to tear or otherwise break apart as the load distribution device moves from the initial configuration to the deformed configuration. As the perforated regions 49 tear apart, the first and second separation portions 51a and 51b are able to move away from each other. It is appreciated that, depending on the amount and location of deformation of the load distribution device 20, it may be the case that not all perforations of all perforated regions 49 tear apart. For instance, it may be the case that not all perforations of all perforated regions tear apart along the transverse direction T to the entire depth of the perforated region 49. It may also be the case that not all perforations of all perforated regions tear apart from the inner surface 33 to the outer surface 35. It may further be the case that no perforations of one or more of the perforated regions 49 tear apart. Respective innermost ones of the perforations of each of the perforated regions 49 along the transverse direction T can define living hinges of the body 32 that allow the openings 46 to widen as the load distribution device 20 moves or flexes from the initial configuration to the deformed configuration, thereby allowing the load distribution device 20 to contour to the epidermis 22 at the adjacent support region 30 (see FIG. 2).

The perforations of the perforated regions 49 can be pre-fabricated prior to use in one example. For instance, the perforations 49 can be defined by cut lines that are produced by laser cutting respective slits into the second surface 32b toward the first surface 32a, and from the outer surface 35 to the inner surface 33. The perforated regions 49 can be elongate along respective axes from the outer surface 35 to the inner surface 33. The axes can be oriented along any suitable directions as desired so long as the openings 46 assist in contouring the load distribution member 20 to conform to the epidermis 22 at the adjacent support locations 30. In one example, the axes can intersect each other at the geometric center 41 of the body 32 as described above. The geometric center 14 of the body 41 can be the geometric center of the outer surface 35, the inner surface 33, or both, in a plane that is oriented perpendicular to the transverse direction T. The load distribution device 20 can have any suitable number of perforated regions as desired, such as three, four, five, six, seven, eight, nine, ten, or greater than ten, such that the openings 46 maintain the structural rigidity of the body 32 while providing for the desired ability for the first surface 32a to conform to the epidermis 22 of the patient 24 at the adjacent support location 30. When the perforations of the perforated regions 49 tear apart, an opening is formed that can be open at the outer surface 35 and the central aperture 38. It is appreciated that the separation zones 45 can include the openings 46, the perforated regions 49, or a combination of the openings 46 and the perforated regions 49.

Referring now to FIGS. 3B-5C, the load distribution device 20 can be configured to attach to the adjacent support location 30 at the first surface 32a. For instance, the first surface 32a can carry at least one attachment member 36, such as a plurality of attachment members 36, configured to attach to the adjacent support location 30. Thus, at least a portion of the first surface 32a can attach to the epidermis 22 at a location adjacent the region of interest 25. In one example, the attachment member is configured to attach directly to the epidermis 22 at the adjacent support location 30. Alternatively or additionally, the first surface 32a can attach to any suitable structure that, in turn supports or is otherwise attached to the adjacent support location 30.

In one example, the attachment member 36 is configured as an adhesive that is carried by the first surface 32a and is suitable to adhesively attach to the adjacent support location 30. The adhesive can be configured as a glue, a tape, or any suitable alternative adhesive. The adhesive can be a double-sided adhesive so as to attach to both the first surface 32a and the adjacent support location 30. For instance, one side of the adhesive can attach to the first surface 32a, and the other side of the adhesive can be covered by a removable backing. The removable backing can be removed so as to expose the other side of the adhesive, which can then be applied to the adjacent support location 30.

The adhesive can be configured to attach to the epidermis 22. Alternatively or additionally, the epidermis 22 can similarly carry an adhesive that is configured to adhesively attach to the adhesive carried by the first surface 32a, or to the first surface 32a directly. It can be desirable for the attachment member 36 to be non-allergenic or hypo-allergenic, and skin friendly such that the attachment member 36 can be applied to the epidermis 22 and subsequently removed from the epidermis 22 without significant damage.

While the attachment member 36 can be configured as an adhesive in one embodiment, it should be appreciated that first surface 32 can attach to the adjacent support location 30 in accordance with any suitable alternative embodiment as desired. For instance, the attachment member 36 can be configured as a strap that can wrap around the patient, a plurality of one of hooks and loops, or can removably attach to the patient 24 any suitable alternative manner as desired. In one example, hooks and loops can interlock with each other so as to attach the body 32 of the load distribution device 20 to the adjacent support location 30.

In still other examples, the load distribution device 20 can be configured to attach to the support surface 34 or otherwise be supported by support the support surface 34 such that the surgical site 27 is aligned with the aperture 38 along the transverse direction T. Thus, the interior surface 33 can substantially circumscribe the region of interest 25 in the manner described above.

The load distribution device 20 can be dimensioned in the longitudinal direction L and in the lateral direction A any suitable distance as desired, depending on the nature and dimensions of the region of interest 25. In one example, the load distribution device 20 can have a length L1 along the longitudinal direction L that is defined by the outer surface 35. The load distribution device 20 can have a width W along the lateral direction A that is defined by the outer surface 35. The length L1 can be greater than the width W in some examples. In other examples, the length L1 can be substantially equal to the width W (see FIG. 6B). By way of example, the length L1 can be in a range from approximately 2 inches to approximately 10 inches, such as from approximately 3 inches to approximately 9 inches, such as from approximately 4 inches to approximately 8 inches, such as from approximately 5 inches to approximately 7 inches. In one example, the length L1 can be approximately 6 inches. By way of example, the load distribution device 20 can define a ratio of length L1 to first thickness T1 that is in a range from approximately 2:1 to approximately 40:1, such as from approximately 5:1 to approximately 14:1. In one example, the ratio can be approximately 8:1.

By way of example, the width W can be in a range from approximately 1 inch to approximately 7 inches, such as from approximately 2 inches to approximately 6 inches, such as from approximately 3 inches to approximately 5 inches. In one example, the width W can be approximately 4 inches. By way of example, the load distribution device 20 can define a ratio of width W to length L1 that is in a range from approximately 1:5 to approximately 1:1, such as from approximately 3:7 to approximately 1:1. In one example, the ratio can be approximately 2:3. By way of example, the load distribution device 20 can define a ratio of width W to first thickness T1 that is in a range from approximately 6:1 to approximately 28:1, such as from approximately 3:1 to approximately 10:1. In one example, the ratio can be approximately 16:3.

The aperture 38 can define any suitable shape as described above, and any suitable dimensions as desired By way of example, the load distribution device 20 can define a ratio of the width of the aperture 38 to the length of the aperture that is in a range from approximately 1:5 to approximately 1:1, such as from approximately 3:7 to approximately 1:1. In one example, the ratio can be approximately 2:3. The ratio of the length to width can be selected to sufficiently reduce the amount of tension that the load distribution device 20 places on the region of interest 25 along the lateral and longitudinal directions A, L during operation of the load distribution device 20. The interior surface 33 can likewise be spaced from the region of interest 25 any distance as desired that is suitable to reduce the amount of tension that the load distribution device 20 places on the region of interest 25.

The aperture 38 and the exterior surface 35 can define a border that extends normally from the inner surface 33 to the outer surface 35 along a straight line in a plane that is perpendicular to the transverse direction T. By way of example, the border can be in a range from approximately 0.25 inch to approximately 3 inches, such as from approximately 0.5 inch to approximately 2.5 inches, such as from approximately 0.75 inch to approximately 2 inches, such as from approximately 1 inch to approximately 1.5 inches. In one example, the border can be approximately 1.25 inches. It should be appreciated that the dimensions disclosed herein are by way of example and not limitations.

When the load distribution device 20 is applied to the patient, a margin can be defined as a distance from the region of interest 25 to the interior surface 33. The margin can be any distance as desired. In one example, the margin can be substantially equal to the first thickness T1 of the load distribution device 20 along the transverse direction T when the load distribution device is in the relaxed state. It should be appreciated that the load distribution devices 20 can be configured to produce any sized margin as desired depending, for instance, on the nature of the region of interest 25.

In one example, the load distribution device 20 can be manufactured with the aperture 38 as described above. Referring to FIG. 4D, in another example, the load distribution device 20 can define a plurality of pre-defined perforations 42. Each perforation 42 can define a respective perforated region. The perforations 42 can include an innermost perforation and a plurality of outwardly spaced perforations, each of the outwardly spaced perforations surrounding respective inner ones of the perforations 42. The perforations can be evenly spaced or variably spaced from each other. Each perforation 42 can substantially circumscribe the respective perforated region. As a result, a plurality of the perforated regions can be defined that increase in size along an outward direction away from the aperture 38. The perforations 42 can extend from the first surface 32a to the second surface 32b along the transverse direction T. Accordingly, a user can remove one or more perforated regions as desired to enlarge the size of the aperture 38.

As described above, in one example the force distribution device 20 can be configured to protect a region of interest 25 from damage due to resting on a support surface for a prolonged period of time. In one example, the region of interest 25 can be undamaged. In other examples shown in FIGS. 7A-7B, it should be appreciated that the force distribution device 20 can also be applied to a wound 60 to prevent the wound from resting directly on a support surface 34 (see FIG. 1), such that the wound 60 is aligned with the aperture 38. The wound 60 can be surgical wound, a traumatic wound, or as discussed above can be a wound defined during and throughout splint application and wearing. In the context of a surgical wound, the force distribution device can be attached to either or both of the epidermis 22 that surrounds and the post-surgical dressing 39 that covers the surgical wound. It should be appreciated that the wound 60 can be disposed at a bony prominence and/or an epicondyle and/or any alternative weight-bearing location of the patient 24 against the support surface.

It should be further appreciated that a kit of load distribution devices 20 can be provided that are each configured to support the patient 24 at different adjacent support locations that are adjacent respective different regions of interest 25. Respective ones of the load distribution devices 20 of the kit can define at least one different characteristic than others of the load distribution devices 20 of the kit. For instance, the at least one different characteristic can be defined by at least one of the length of the aperture 38, the width of the aperture 38, the thickness of the body 32, the length of the load distribution device 20, the width of the load distribution device 20, the relaxed thickness of the load distribution device 20, the material of the load distribution device 20, the number of perforations 42, the type of attachment member 36, and the porosity of the body 32. For instance, different ones of the bodies 32 of the kit can have porosities greater than or less than others of the bodies 32 of the kit. The kit can further include a plurality of load distribution devices 20 having the same characteristics, such that one of the load distribution devices 20 can replace a discarded load distribution device.

It should be appreciated that a method is provided that isolates the anatomical forces of the patient 24 from the region of interest 25. The method can include the step of identifying one or more vulnerable regions of interest of a patient that can be supported by a support surface for a prolonged period of time sufficient to potentially damage the region of interest, particularly when the patient is unable to sense discomfort and/or communicate the discomfort to the care giver. The damage can include a wound such as a bed sore, nerve damage, soft tissue damage, or any alternative damage that can result. The method can include the next step of placing one or more load distribution devices 20 between the patient 24 and the support surface 34 such that the respective one or more regions of interest are aligned with the apertures 38 of the load distribution devices 20, respectively, as described above. The placing step can further include the step of attaching the first surface 32a to the adjacent support location 30. For instance, the method can include the step of adhesively attaching the first surface 32a to the epidermis 22 at the adjacent support location 30.

The method can further include the step of iterating the patient 24 to a desired position against the support surface 34, such that the anatomical forces of the region(s) of interest 25 are isolated from the support surface 34, for instance with respect to the transverse direction T. The transverse direction T can be defined by the vertical direction, for instance, when the support surface 34 is defined by a bed, couch, cot, or the like. The transverse direction T can be defined by the horizontal direction or a direction offset to each of the vertical direction and the horizontal direction, for instance, when the support surface 34 is defined by a chair or recliner. Thus, the region of interest 25 is unloaded by directly decreasing the pressure that is applied to the region of interest 25 from the support surface 34. The method can further include the step of compressing the load distribution device 20 between the patient 24 and the support surface 34 under the force of the patient 24. The force of the patient can be defined by either or both of the weight of the patient at the region of interest 25 and muscular bias of the region of interest 25 against the support surface 34. The method can further include the step of conforming the first surface 32a to the supine patient 24, for instance by causing the body 32 to flex about the living hinges defined by the openings 46.

The method can further include the step of removing the load distribution device 20 from the patient 24. The method can then include the step of re-attaching the previously-removed load distribution device 20 or a new load distribution device between the patient 24 to the adjacent support location 30 in the manner described above, and positioning the load distribution device 20 against the support surface in the desired manner.

The embodiments described in connection with the illustrated embodiments have been presented by way of illustration, and the present invention is therefore not intended to be limited to the disclosed embodiments. Furthermore, the structure and features of each the embodiments described above can be applied to the other embodiments described herein. Accordingly, those skilled in the art will realize that the invention is intended to encompass all modifications and alternative arrangements included within the spirit and scope of the invention, as set forth by the appended claims.

What is claimed:

1. A method of isolating anatomical forces of an identified vulnerable region of interest of a patient from a support surface, the method comprising the step of:
placing a load distribution device between the patient and the support surface such that an entirety of the region of interest is aligned with an aperture that extends at least into a body of the load distribution device, wherein the placing step comprises aligning the body with an adjacent support location that is adjacent the region of interest; and
iterating the patient to a position against the support surface such that the load distribution device isolates anatomical forces of the patient between the region of interest and the support surface.

2. The method of claim 1, wherein the placing step comprises flexing the body about living hinges that are defined by the body so as to conform an inner surface of the body against the patient at the adjacent support location.

3. The method of claim 2, wherein the flexing step comprises flexing the body about bases of openings that extend into the body from an outer surface that is opposite the inner surface toward the inner surface without extending through the inner surface.

4. The method of claim 3, wherein the openings extend from an interior surface of the body that defines the opening to an exterior surface of the body that is opposite the inner surface.

5. The method of claim 4, wherein the load distribution device comprises at least four openings.

6. The method of claim 1, wherein the placing step comprises flexing the body, which causes perforations of perforated regions of the body to break apart.

7. The method of claim 1, wherein the placing step comprises adhesively attaching the load distribution device to the adjacent support location.

8. The method of claim 1, further comprising the step of compressing the load distribution device between the patient and the support surface under the weight of the patient.

9. The method as recited in claim 1, wherein the region of interest is undamaged prior to the placing step.

10. The method of claim 1, wherein the region of interest comprises either or both of a bony prominence and an epicondyle.

11. The method of claim 10, wherein the iterating step causes the region of interest to extend into the aperture in a direction from the first surface toward the second surface, such that the region of interest terminates at a location in the aperture between the first surface and the second surface.

12. The method of claim 1, wherein the aperture extends entirely through the body of the load distribution device.

13. A load distribution device configured to support a patient that is supported by a support, the load distribution device comprising:
a compressible body having a first surface configured to face the patient, and a second surface opposite the first surface and configured to face the support surface,
wherein 1) the load distribution device defines an internal surface that defines an aperture that extends at least into the body from the first surface toward the second surface, 2) the load distribution device defines greater than two separation zones that extend into the second surface toward the first surface, wherein each of the separation zones extends from the internal surface to an external surface opposite the internal surface, and
wherein the load distribution device is configured to be placed against the patient such that a vulnerable region of interest is in alignment with the aperture so as to isolate an anatomical load between the region of interest and the support surface.

14. The load distribution device of claim 13, wherein the separation zones define respective openings that extend to a depth from the second surface toward the first surface.

15. The load distribution device of claim 14, wherein the openings terminate at respective bases that define living hinges, and the load distribution device is configured to flex about the living hinges as the load distribution device conforms to an adjacent support location that is adjacent the region of interest.

16. The load distribution device of claim 15, wherein the openings are elongate along respective axes from the internal surface to the external surface, and the axes intersect each other.

17. The load distribution device of claim 16, wherein the axes intersect each other at a geometric center of the load distribution device.

18. The load distribution device of claim 13, wherein the separation zones define perforated regions that have perforations that are configured to break apart as the body iterates from an initial configuration to a deformed configuration.
